(19) United States
(12) Patent Application Publication
(10) Pub. No.: US 2023/0190612 A1
(43) Pub. Date: Jun. 22, 2023

(54) METHODS AND COMPOSITIONS FOR THE PROTECTION OF HAIR

(71) Applicant: Gelest, Inc., Morrisville, PA (US)

(72) Inventors: Tatyana ABEL-ROBERMAN, Bryn Athyn, PA (US); Barry C. ARKLES, Pipersville, PA (US); Alison Anne PHILLIPS, Feasterville, PA (US)

(21) Appl. No.: 17/555,908

(22) Filed: Dec. 20, 2021

(51) Int. Cl. A61K 8/58; A61Q 5/00; A61K 8/36; A61K 8/362; A61K 8/49; A45D 7/06

(52) U.S. Cl. CPC A61K 8/585; A61Q 5/002; A61K 8/36; A61K 8/362; A61K 8/4973; A45D 7/06

(57) ABSTRACT

A method of protecting hair from damage by applying a long-chain unhydrolyzed alkyltrialkoxysilane to hair shafts and then hydrolyzing and polymerizing the alkyltrialkoxysilane to form a non-penetrating outer layer on the hair shaft by utilizing pH adjusted water followed by the application of heat. Alternatively, the alkyltrialkoxysilane may be applied to the hair in combination with an alkyl or alkenyl succinic anhydride prior to the application of heat.

### BACKGROUND OF THE INVENTION

Hair damage due to physical stress, chemical treatment, or natural aging is naturally unpreventable. Since hair does not restore itself, damage is typically irreversible and most hair treatments concentrate on fixing already damaged hair.

The outermost layer of the hair shaft, the hair surface structure, is rich in 18-methyleicosanoic acid (18-MEA) that is covalently bound to the keratin-associated proteins of the hair cuticle, creating a relatively lubricious hydrophobic protective overlay (see Tokunaga, Cosmetics, 6, 31 (2019)). If the layer of 18-MEA is lost, it is not replenished, and the natural structure and behavior of the hair is compromised. Concomitant with the loss, negatively charged sulfonates (SO₃⁻) become active species that are exposed on the outermost hair surface and cause hair to become relatively hydrophilic, sensitive to changes in humidity, and to appear dull and frizzy. Loss of 18-MEA also exposes keratin-associated proteins, peptides, and amino acids, which leads to their further damage and destruction.

The loss of 18-MEA from the hair is traditionally addressed with products containing positively charged quaternary salts which ionically associate with the SO₃⁻ sites and temporarily neutralize the negative charge of the hair. The major downside of the use of these salts is that they are only weakly associated with the damaged sites of the hair shaft and are limited to low concentration levels. Additionally, the ionic bond between a quaternary salt and SO₃⁻ is easily broken by washing or simply brushing the hair, making their effects short-lived. Most products which include quaternary salts to treat 18-MEA loss incorporate fatty acids and other ingredients that create build-up when overused. Consequently, most of these treatments are temporary and therefore must be used regularly to achieve their benefits. These products additionally do not prevent the further loss of 18-MEA, but only reduce the appearance of existing damage due to charge neutralization.

Other previous approaches to treating damaged hair have been to modify damaged hair by penetrating the cuticle of the hair that has been depleted of 18-MEA. For example, U.S. Pat. No. 8,470,064 teaches that short chain alkyltrialkoxysilanes with six or fewer carbons are able to penetrate the cuticle of the hair and improve manageability of the hair. Regardless of the benefits, a structural change occurs to the hair treated by these methods. Methods of hair treatment in which pre-hydrolyzed or pre-polymerized alkyltrialkoxysilanes (alkysilanetriols or alkylsilsesquioxanes) are applied to the hair, either directly or stabilized by emulsions, are described in U.S. Pat. No. 10,487,242. However, there remains a need in the art for methods for preventing damage to the hair shaft.

### BRIEF SUMMARY OF THE INVENTION

In one aspect of the disclosure, provided is a method of protecting hair comprising:
(a) applying at least one long chain alkyltriethoxysilane onto wet or dry hair;
(b) applying water adjusted to an acidic pH to the hair; and
(c) heating the hair.

In another aspect of the disclosure, provided is a method of protecting hair comprising:
(a) applying a mixture comprising at least one long chain alkyltriethoxysilane and at least one long chain alkyl or alkenyl succinic anhydride onto wet or dry hair; and
(b) heating the hair.

In a further aspect of the disclosure, provided is a composition comprising at least one long chain alkyltriethoxysilane and at least one long chain alkyl or alkenyl succinic anhydride, wherein the composition forms a protective, elastic outer layer when applied to hair and dried.

### DETAILED DESCRIPTION OF INVENTION

Aspects of the disclosure relate to methods for protection of the hair shaft prior to damage, thus retaining the original structure and prolonging the life span of hair. Such methods provide additional benefits by providing healthier appearance, strength, and shine when used on either damaged or undamaged hair.

By employing the methods described herein, a protective, elastic, non-penetrating outer layer is created on individual hair shafts which prevents the of loss of 18-MEA during chemical treatments, physical processing, and aging, and provides hair shaft integrity. This is achieved by applying to wet or dry hair alkyltriethoxysilanes with a sufficient number of carbon atoms such that penetration of the cuticle does not occur and the structural integrity of the hair shaft is maintained. After application, the alkyltriethoxysilane undergoes a hydrolysis and polymerization process on the outer surface of the hair shafts upon changing the pH from neutral to moderately acidic and applying heat. The appropriate alkyl group length, about 8 to about 22 carbon atoms, is selected both because hydrophobic behavior is similar to 18-MEA and because the molecular dimensions are large enough to prevent penetration into the hair shaft. Accordingly, the alkyl group may have about 8, 9, 10, 11, 12, 13, 14, 15, 16, 17, 18, 19, 20, 21, or 22 carbon atoms. The compatibility and performance of alkyltriethoxysilanes having about 12 to about 16 carbon atoms appears optimal and is presently preferred. Accordingly, preferred alkyltriethoxysilanes contain about 12, 13, 14, 15, or 16 carbon atoms. Once applied to the hair, hydrolyzed, and polymerized, a protective layer forms and the hair fibers remain as individual strands, i.e., they are not attached or connected by a continuous film, but rather manifest a smooth cohesive behavior and are better able to resist mechanical damage.

A first method for protecting hair according to the disclosure comprises applying at least one long chain alkyltriethoxysilane directly onto wet or dry hair, followed by applying to the hair water adjusted to an acidic pH, preferably a pH of about 4-5, and heating the hair. The term “long chain alkyltriethoxysilane” may be understood to encompass triethoxysilanes in which the carbon chain in the alkyl group contains about 8 to about 22 carbon atoms, more preferably about 12 to about 16 carbon atoms, as previously described. The hydrophobic alkyl portion of the alkyltriethoxysilane forms a layer by hydrophobic interaction with the hydrocarbon chains of 18-MEA. After the alkyltriethoxysilane is applied to the hair, it rapidly associates with the 18-MEA-rich outer layer and the triethoxy part of the silane is hydrolyzed by the application of the acidic water. This initiates hydrolysis of the silane, which then condenses and polymerizes upon application of heat using heating tools commonly found in a hair salon, such as a blow dryer, curling iron, and/or flat iron. The pH adjustment of the water may be accomplished by well-known methods, such as by forming dilute solutions of acetic acid, citric acid, or other acidic species, or by employing buffered systems, such as sodium acetate/acetic acid, so that such dilute solutions may be applied to the hair in the second step. Alternatively, the method may comprise first wetting the hair with water adjusted to an acidic pH, preferably a pH of about 4-5, and then applying at least one long chain alkyltriethoxysilane. However, this method requires more vigorous application to achieve uniform behavior.

A second method according to the disclosure for protecting hair comprises applying to the wet or dry hair a mixture containing primarily at least one long chain alkyltriethoxysilane and at least one long chain alkyl or alkenyl succinic anhydride. The term “long chain alkyl or alkenyl succinic anhydride” may be understood to encompass succinic anhydrides in which the carbon chain in the alkyl or alkenyl group contains about 8 to about 18 carbon atoms, more preferably about 16 carbons. Accordingly, the alkyl or alkenyl group may contain about 8, 9, 10, 11, 12, 13, 14, 15, 16, 17, or 18 carbon atoms. The amount of succinic anhydride in the mixture is preferably at least about 0.5 wt % and less than about 10 wt %, more preferably less than about 2 wt %. The amount of triethoxysilane in the mixture is preferably about 50 wt % to about 99.5%. Thus, the mixture may contain only succinic anhydride and triethoxysilane (0.5 wt % to 10 wt % succinic anhydride and balance triethoxysilane) or may be a solution containing the two components in the preferred concentrations with balance water or a solvent as previously described This method eliminates the requirement of a separate application step of acidic water and the hair can be wetted before or after application. That is, the mixture may be applied to wet or dry hair. Following application of the mixture containing alkyltriethoxysilane and the alkyl or alkenyl succinic anhydride, the components are further condensed by applying heat to the hair using heating tools commonly found in a hair salon.

It is also within the scope of the disclosure to apply the long-chain alkyltrialkoxysilanes to the hair in combination with known carriers or vehicles that do not hydrolyze the alkyltrialkoxysilanes prior to application to the hair. For example, formulations containing 5-60 wt % of the alkyltrialkoxysilane may be mixed with compatible non-volatile solvents, such as, but not limited to, 2-ethylhexylpalmitate or octyltrimethicone, or with volatile solvents, such as, but not limited to, propyltrimethicone or decamethylcyclopentasiloxane. Such solvents are appropriate when the alkoxysilane is administered alone or when mixed with the succinic anhydride, provided that the components are present in the solution in the desired amounts.

Hair treated by the methods according to the disclosure is protected from damage (such as environmental, physical stress, chemical treatment, and natural aging) and maintains the behavior of “healthy” hair, including combability, gloss, reduction of frizz, and smoothness of feel. Further, as a result of the non-penetrating outer layer formed on individual shafts, the protected individual strands of hair can move independently, yet, because they have similar physical properties, are able to move in a relatively cohesive manner, maintaining natural look and behavior. This is in stark contrast to the application of polymeric materials such as oligomeric condensation products of alkoxysilanes to hair, which leads to bridging between hair shafts. Such bridging does not occur to hair treated by the methods described herein.

The methods described herein are appropriate for treating all types of hair, including hair which is straight, wavy, curly, or kinky, hair which is virgin or has been bleached, colored, permed, relaxed, or undergone any other type of chemical or physical treatment, and hair of any form, shape, or length.

The invention will now be described in connection with the following non-limiting examples.

### EXAMPLE 1: APPLICATION OF N-OCTYLTRIETHOXYLSILANE TO VIRGIN HAIR WITH ACIDIC WATER AND/OR HEAT EXPOSURE

Sample Preparation: A virgin tress with frizzy, and kinky hair (curl pattern type IV) 4 inches wide and 6 inches long (purchased from IHIP) was washed with generic shampoo, air-dried, and cut into four strips of 1 inch each labeled A, B, C, and D.

Sample 1—Hair tress A weighing 4.0 g was washed with shampoo and conditioner and left to air dry. This sample is referred to as the control.

Sample 2—Hair tress B was sprayed with n-octyltriethoxysilane (CAS #2943-75-1) to full saturation. Using fingers, the material was spread throughout the tress to cover the entire surface of the hair shafts. The treated hair tress was exposed to air at room temperature for 10 minutes. After 10 minutes, water adjusted to pH 4.5 with acetic acid was sprayed onto the tress. The tress was covered in plastic for 30 minutes, then dried with a hair dryer on high speed and high heat. When the tress was completely dry, it was combed and flat ironed at 410° F. in 0.5-inch sections using 5-7 passes. The tress was washed with shampoo and conditioner after 24 hours and air-dried at room temperature with a relative humidity of 67%. Treated hair showed increased shine, substantial reduction in frizz, and increased smoothness, which aided in dry combing.

Sample 3—Hair tress C was sprayed with water adjusted to pH 4.5 with acetic acid until the hair was saturated. The hair tress was then sprayed with sufficient n-octyltriethoxysilane to saturate the hair. Using fingers, the material was spread onto the tress to distribute over the entire surface of the hair shaft. The tress was covered in plastic for 30 minutes, dried with a hair dryer on high speed and high heat, then combed and flat ironed at 410° F. in 0.5-inch sections using 5-7 passes. The tress was washed with shampoo and conditioner after 24 hours and air-dried at room temperature with a relative humidity of 67%. Treated hair showed increased shine, substantial reduction in frizz, and increased smoothness, which aided in dry combing.

Sample 4—Hair tress D was sprayed with n-octyltriethoxysilane. Using fingers, the material was spread onto the tress to distribute over the entire surface of the hair shaft. The tress was allowed to react for 30 minutes covered in plastic, dried with a hair dryer on high speed and high heat, then combed and flat ironed at 410° F. in 0.5-inch sections using 5-7 passes. The tress was washed with shampoo and conditioner after 24 hours and air-dried at room temperature with a relative humidity of 67%. Treated hair showed increased shine, substantial reduction in frizz, and increased smoothness, which aided in dry combing.

### Analysis of Hair Tresses A-D

Hair tresses A through D were laid flat on a surface and a measurement was taken at the widest area. The width measurements of the treated hair were compared to the control (A) as an indication of frizz, with a wider hair tress corresponding to more frizz. Hair tress A (control) had a width of 13 cm. Hair tress B had a width of 7.5 cm, corresponding to a 42% reduction of frizz. Hair tress C had a width of 7.7 cm, corresponding to 40% reduction of frizz. Hair tress D had a width of 7.0 cm, corresponding to 46% reduction of frizz.

Samples B, C, and D showed defined curl, increased shine, and increased smoothness compared to Sample A (control); these properties were assessed and evaluated by individuals with expertise in the cosmetic industry. The following relationships were observed:
Smoothness: C>B>D,
Shine: D=C=B

SEM images were collected of samples A, B, C, and D. Samples B, C, and D showed the presence of silicone on the outer layer of the hair shaft by Energy Dispersive X-ray Spectroscopy (EDS) analysis.

### EXAMPLE 2: APPLICATION OF HEXADECYLTRIETHOXYSILANE TO BLEACHED HAIR WITH ACIDIC WATER AND/OR HEAT EXPOSURE

Sample Preparation: A hair tress with virgin, frizzy, and kinky hair (curl pattern type IV) 4 inches wide and 6 inches long (purchased from IHIP) was bleached twice with level 9 bleach using 40 volume developer. The hair tress was washed with generic shampoo, air-dried, and cut into four strips of 1 inch each labeled A, B, C, and D.

Sample 1—Hair tress A was washed with shampoo and conditioner and left to air dry. This sample is referred to as the control.

Sample 2—Hair tress B was sprayed with hexadecyltriethoxysilane (CAS #16415-13-7) to full saturation. Using fingers, the material was spread onto the tress to cover the entire surface of the hair shaft. The treated hair tress was exposed to air at room temperature for 10 minutes. After 10 minutes, water adjusted to pH 4.5 with acetic acid was sprayed onto the tress. The hair tress was left to react for 30 minutes covered in plastic, then dried with a hair dryer on a high speed and high heat. When the tress was completely dried, it was combed and flat ironed at 410° F. in 0.5-inch sections using 5-7 passes. The tress was washed with shampoo and conditioner after 24 hours and air-dried at room temperature with a relative humidity of 65%. Treated hair showed increased shine, substantial reduction in frizz, and increased smoothness, which aided in dry combing.

Sample 3—Hair tress C was sprayed with water adjusted to pH 4.5 with citric acid to full saturation, then sprayed with hexadecyltriethoxysilane in an amount sufficient to saturate the hair. Using fingers, the material was spread onto the tress to cover the entire surface of the hair shaft. The tress was left to react for 30 minutes covered in plastic, dried with a hair dryer on high speed and high heat, then combed and flat ironed at 410° F. in 0.5-inch sections using 5-7 passes. The tress was washed with shampoo and conditioner after 24 hours and air-dried at room temperature with a relative humidity of 65%. Treated hair showed increased shine, substantial reduction in frizz, and increased smoothness, which aided in dry combing.

Sample 4—Hair tress D was sprayed with hexadecyltriethoxysilane (CAS #16415-13-7) until the hair was saturated. Using fingers, the material was spread onto the tress to cover the entire surface of the hair shaft. The hair tress was left to react for 30 minutes covered in plastic, dried with a hair dryer on high speed and high heat, then combed and flat ironed at 410° F. in 0.5-inch sections using 5-7 passes. The tress was washed with shampoo and conditioner after 24 hours and air-dried at room temperature with a relative humidity of 65%. Treated hair showed increased shine, substantial reduction in frizz, and increased smoothness, which aided in dry combing.

### Analysis of Hair Tresses A-D

Hair tresses A through D were laid flat on a surface and a measurement was taken at the widest area. The width measurements of the treated hair were compared to the control as an indication of frizz with a wider hair tress corresponding to more frizz. Hair tress A (control) had a width of about 11 cm. Hair tress B had a width of about 4.5 cm, corresponding to a 59% reduction of frizz. Hair tress C had a width of about 5.0 cm, corresponding to a 55% reduction of frizz. Hair tress D had a width of about 8.0 cm, corresponding to a 27% reduction of fizz.

Smoothness and shine were assessed and evaluated by individuals with expertise in the cosmetic industry. The following relationships were observed: samples B, C, and D demonstrated a straightening effect compared to Sample A (control). Samples B, C, and D also showed increased shine and smoothness.

SEM images were collected of samples A, B, C, and D. Samples B, C, and D and showed the presence of silicone on the outer layer of the hair shaft by Energy Dispersive X-ray Spectroscopy (EDS) analysis.

### EXAMPLE 3: APPLICATION OF HEXADECYLTRIETHOXYSILANE APPLIED TO HAIR WITH LONG-CHAIN ALKYL SUCCINIC ANHYDRIDE APPLICATION AND HEAT EXPOSURE

Sample Preparation: A virgin tress with frizzy, and kinky hair (curl pattern type IV) 4 inches wide and 6 inches long (purchased from IHIP) was washed with generic shampoo, air-dried, and cut into four strips of 1 inch each labeled A, B, C, and D.

Sample 1—Hair tress A weighing 4.0 g was washed with shampoo and conditioner and left to air dry. This sample is referred to as the control.

Sample 2—Hair tress B was saturated with water and then sprayed with a solution composed of 99% hexadecyltriethoxysilane (CAS #16415-13-7) and 1% dodecenylsuccinic anhydride. Using fingers, the material was spread onto the wet tress to cover the entire surface of the hair shaft. The hair tress was left to react for 30 minutes covered in plastic, dried with a hair dryer on high speed and high heat, then combed and flat ironed at 410° F. in 0.5-inch sections using 5-7 passes. The tress was washed with shampoo and conditioner after 24 hours and air-dried at room temperature with a relative humidity of 67%. Treated hair showed increased shine, substantial reduction in frizz, and increased smoothness, which aided in dry combing.

Sample 3—Hair tress C was saturated with water and then sprayed with a solution composed of 99% hexadecyltriethoxysilane (CAS #16415-13-7) and 1% hexadecenylsuccinic anhydride. Using fingers, the material was spread onto the wet tress to cover the entire surface of the hair shaft. The hair tress was left to react for 30 minutes covered in plastic, dried with a hair dryer on high speed and high heat, then combed and flat ironed at 410° F. in 0.5-inch sections using 5-7 passes. The tress was washed with shampoo and conditioner after 24 hours and air-dried at room temperature with a relative humidity of 67%. Treated hair showed increased shine, substantial reduction in frizz, and increased smoothness, which aided in dry combing.

Sample 4—Hair tress D was saturated with water and then sprayed with a solution composed of 99% hexadecyltriethoxysilane (CAS #16415-13-7) and 1% octadecenylsuccinic anhydride (CAS #28777-98-2). Using fingers, the material was spread onto the wet tress to cover the entire surface of the hair shaft. The hair tress was left to react for 30 minutes covered in plastic, dried with a hair dryer on high speed and high heat, then combed and flat ironed at 410° F. in 0.5-inch sections using 5-7 passes. The tress was washed with shampoo and conditioner after 24 hours and air-dried at room temperature with a relative humidity of 67%. Treated hair showed increased shine, substantial reduction in frizz, and increased smoothness, which aided in dry combing.

### Analysis of Hair Tresses A-D

Hair tresses A through D were laid flat on a surface and a measurement was taken at the widest area. The width measurements of the treated hair were compared to the control (A) as an indication of frizz with a wider hair tress corresponding to more frizz. Hair tress A (control) had a width of 13 cm. Hair tress B had a width of about 7 cm, corresponding to a 46% reduction of fizz. Hair tress C had a width of about 5 cm, corresponding to a 61.5% reduction of frizz. Sample D had a width of about 5.5 cm, corresponding to a 57% reduction.

Smoothness and shine were assessed and evaluated by individuals with expertise in the cosmetic industry. The following relationships were observed: sample C demonstrated a straightening effect compared to Sample A (control). Sample C also showed increased shine and smoothness.

### EXAMPLE 4: APPLICATION OF VARIOUS CHAIN LENGTH ALKYLTRIETHOXYLSILANES AND ACIDIC WATER TO HAIR FOLLOWED BY HEAT EXPOSURE

Sample Preparation: A virgin tress with frizzy, and kinky hair (curl pattern type IV) 4 inches wide and 6 inches long (purchased from IHIP) was washed with generic shampoo, air-dried, and cut into four strips of 1 inch each labeled A, B, C, and D.

Sample 1—Hair tress A was washed with shampoo and conditioner and left to air dry. This sample is referred to as the control.

Sample 2—Hair tress B was sprayed with water adjusted to pH 4.5 with citric acid until the hair was saturated, then the hair tress was sprayed with n-octyltriethoxysilane (CAS #2943-75-1) to full saturation. Using fingers, the material was spread onto the tress to distribute over the entire surface of the hair shaft. The tress was allowed to react for 30 minutes covered in plastic, dried with a hair dryer on high speed and high heat, then combed and flat ironed at 410° F. in 0.5 inch sections using 5-7 passes. The tress was washed with shampoo and conditioner after 24 hours and air-dried at room temperature with a relative humidity of 67%. Treated hair showed increased shine, substantial reduction in frizz, and increased smoothness, which aided in dry combing.

Sample 3—Hair tress C was sprayed with water adjusted to pH 4.5 with citric acid until the hair was saturated, then the hair tress was sprayed with isooctyltriethoxysilane to full saturation. Using fingers, the material was spread onto the tress to distribute over the entire surface of the hair shaft. The tress was allowed to react for 30 minutes covered in plastic, dried with a hair dryer on high speed and high heat, then combed and flat ironed at 410° F. in 0.5 inch sections using 5-7 passes. The tress was washed with shampoo and conditioner after 24 hours and air-dried at room temperature with a relative humidity of 67%. Treated hair showed increased shine, substantial reduction in frizz, and increased smoothness, which aided in dry combing.

Sample 4—Hair tress C was sprayed with water adjusted to pH 4.5 with citric acid until the hair was saturated, then the hair tress was sprayed with hexadecyltriethoxysilane to full saturation. Using fingers, the material was spread onto the tress to distribute over the entire surface of the hair shaft. The tress was allowed to react for 30 minutes covered in plastic, dried with a hair dryer on high speed and high heat, then combed and flat ironed at 410° F. in 0.5 inch sections using 5-7 passes. The tress was washed with shampoo and conditioner after 24 hours and air-dried at room temperature with a relative humidity of 67%. Treated hair showed increased shine, substantial reduction in frizz, and increased smoothness, which aided in dry combing.

### Analysis of Hair Tresses A-D

Hair tresses A through D were laid flat on a surface and a measurement was taken at the widest area. The width measurements of the treated hair were compared to the control as an indication of frizz with a wider hair tress corresponding to more frizz. Hair tress A (control) had a width of 12 cm. Hair tress B had a width of 6.8 cm, corresponding to a 43% reduction of frizz. Hair tress C had a width of 6.0 cm, corresponding to 50% reduction of frizz. Hair tress D had a width of 5.1 cm, corresponding to 58% reduction of frizz.

Smoothness and shine were assessed and evaluated by individuals with expertise in the cosmetic industry. The following relationships were observed.

Samples B, C, and D show increased shine and increased smoothness compared to Sample A (control). Smoothness: C>D>B, Shine: D=C=B.

SEM images were collected of samples A, B, C, and D. Samples B, C, and D showed the presence of silicone on the outer layer of the hair shaft by EDS analysis.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of protecting hair comprising:
(a) applying at least one long chain alkyltriethoxysilane onto wet or dry hair,
(b) applying water adjusted to an acidic pH to the hair; and
(c) heating the hair.

2. The method according to claim 1, wherein the alkyl group in the at least one alkyltriethoxysilane contains about 8 to about 22 carbon atoms.

3. The method according to claim 2, wherein the alkyl group in the at least one alkyltriethoxysilane contains about 12 to about 16 carbon atoms.

4. The method according to claim 1, wherein the water in step (b) has a pH of about 4-5.

5. The method according to claim 1, wherein step (c) comprises applying heat from a blow dryer, a curling iron, and/or a flat iron.

6. The method according to claim 1, wherein the protected hair has a protective, elastic outer layer.

7. The method according to claim 1, wherein the alkyltriethoxysilane is hydrolyzed and polymerized on a surface of the hair to form a protective coating.

8. The method according to claim 1, wherein step (b) comprises applying to the hair a solution of acetic acid, citric acid, or a buffered system.

9. The method according to claim 1, wherein step (a) comprises applying a solution containing about 5 to 60 wt % of the at least one alkyltriethoxysilane in a volatile or non-volatile solvent.

10. A method of protecting hair comprising:
(a) applying a mixture comprising at least one long chain alkyltriethoxysilane and at least one long chain alkyl or alkenyl succinic anhydride onto wet or dry hair; and
(b) heating the hair.

11. The method according to claim 10, wherein the alkyl group in the at least one alkyltriethoxysilane contains about 8 to about 22 carbon atoms.

12. The method according to claim 11, wherein the alkyl group in the at least one alkyltriethoxysilane contains about 12 to about 16 carbon atoms.

13. The method according to claim 10, wherein the alkyl or alkenyl group in the alkyl or alkenyl succinic anhydride contains about 8 to about 18 carbon atoms.

14. The method according to claim 10, wherein step (b) comprises applying heat from a blow dryer, a curling iron, and/or a flat iron.

15. The method according to claim 10, wherein the protected hair has a protective, elastic outer layer.

16. The method according to claim 10, wherein the alkyltriethoxysilane is hydrolyzed and polymerized on a surface of the hair to form a protective coating.

17. The method according to claim 10, wherein the mixture contains at least about 0.5 wt % and less than about 10 wt % of the at least one long chain alkyl or alkenyl succinic anhydride and balance the at least one alkyltriethoxysilane.

18. The method according to claim 10, wherein the mixture contains at least about 0.5 wt % and less than about 10 wt % of the at least one long chain alkyl or alkenyl succinic anhydride and about 5 to 60 wt % of the at least one alkyltriethoxysilane.

19. A composition comprising at least one long chain alkyltriethoxysilane and at least one long chain alkyl or alkenyl succinic anhydride, wherein the composition forms a protective, elastic outer layer when applied to hair and dried.

20. The composition according to claim 19, wherein the alkyl group in the at least one alkyltriethoxysilane contains about 8 to about 22 carbon atoms and the alkyl or alkenyl group in the at least one alkyl or alkenyl succinic anhydride contains about 8 to about 18 carbon atoms.
